(19) United States
(12) Patent Application Publication
(10) Pub. No.: US 2014/0113316 A1
(43) Pub. Date: Apr. 24, 2014

(54) DEVICE, METHOD AND KIT FOR THE DETECTION OF DIFFERENT MARKERS IN DIFFERENT CELLULAR OR MOLECULAR TYPES AND THEIR QUANTIFICATIONS

(71) Applicant: Alessandra Mazzeo, Campobasso (IT)

(72) Inventor: Alessandra Mazzeo, Campobasso (IT)

(21) Appl. No.: 14/058,946

(22) Filed: Oct. 21, 2013

Related U.S. Application Data
(63) Continuation of application No. PCT/IB2012/052021, filed on Apr. 23, 2012.

(30) Foreign Application Priority Data
Apr. 21, 2011 (IT) CS2011A000012
Apr. 20, 2012 (IT) CS2012A000019

(51) Int. Cl. G01N 33/569

(52) U.S. Cl. CPC G01N 33/5695
USPC 435/7.24; 435/287.2; 435/7.1; 435/7.92

(57) ABSTRACT

Microplates or microstrips having wells with extended length to receive immunosorbent elements protruding from a rod at the same modular distance of wells arranged in standard microstrips or microplates and methods of use are provided. A solid phase is constituted by the extended wells, each of which immobilize a different type of cell or molecule in the sample; the other solid phase is constituted by immunosorbent elements protruding from a rod, each of which has been previously coated with one of the same markers to be detected in the sample. Ligands for markers to be detected are added in a liquid phase to the wells; allowing the ligands competitively bind to the immunosorbent elements, inversely proportional to the quantity of the markers of each type of cell or molecule immobilized on the proper extended well. These ligands are simultaneously quantifiable by an immunoenzymatic assay using chromogenic substrate and a spectrophotometer.

### CROSS REFERENCE TO RELATED APPLICATION

This application is a continuation of prior filed International Application No. PCT/IB2012/052021, as filed on Apr. 23, 2012, entitled “Device, Method And Kit For The Detection Of Different Markers In Different Types Of Cells Or Molecules And Their Quantification”, which claims priority to Italian Application No. CS2011A000012, as filed on Apr. 21, 2011, and Italian Application No. CS2012A000019, as filed on Apr. 20, 2012. The contents of which are herein incorporated by reference.

### BACKGROUND OF THE INVENTION

The indirect diagnosis of tuberculosis, caused by Mycobacterium tuberculosis, one of the most worldwide spread infection among the human population, cannot be carried out by the standard serological tests and it is based on the cellular response detection methods, such as:
in vivo skin test, that has to be read after 72 hours, which is useful to detect infection cases regardless of the mycobacterium localization, based on a IV type delayed hypersensitivity response in the infected host, subsequent to the in vivo mycobacterium tuberculosis hapten inoculation;
gamma-interferon test in blood sample, commercially available, that have to be read after 18-24 hours; it detects the interleukins, in particular the gamma-interferon one, released by TH1-lymphocytes cooperating in cell-mediated response, if previously sensitized by the tuberculosis infection, when they are in culture and are stimulated by mycobacterial antigens.

Differentiating the human active tuberculosis from the latent one is of utmost importance for a focused therapy that takes, respectively, 9 or 6 months. Furthermore, it is useful for the monitoring of the infection during the therapy; it is based on the TH1-lymphocytes number detection—that are sensitized against specific tuberculosis antigens such as the ESAT-6, CFP-10, TB7.7—which secrete gamma-interferon and other interleukins when stimulated by antigens put in the culture medium.

In order to solve the problem concerning the time-spending analytical methods based on lymphocytes culturing, the direct lymphocytes markers detection and quantification are proposed, being that markers necessary expressed on the lymphocyte membrane when stimulated by a specific antigen to produce interleukins.

The kit arrangement structured in order to have a rapid, time-saving and minimal equipment requiring screening or diagnostic kit, concerning the human tuberculosis, could be of great help in worldwide health programmes, in sanitary alerts, in sanitary control where large populations move from highly infected areas towards tuberculosis-free areas; furthermore, the kit would be useful in minimal setting countries, where the lacking of structural and infrastructural facilities do not allow the extensive monitoring of infected people by the commercially available diagnostic kits.

The bovine tuberculosis, caused by Mycobacterium bovis, is regulated by statutory eradication plans due to the serious pathologies induced in herds and due to its importance as zoonosis. Due to the fact that the indirect diagnosis cannot be performed by the standard serological methods, this is based on cellular response detection methods, such as:
in vivo skin test, that have to be read after 72 hours, which is useful to detect infection cases regardless of the mycobacterium localization, based on a IV type delayed hypersensitivity response in the infected host, subsequent to the in vivo mycobacterium tuberculosis hapten inoculation;
gamma-interferon test in blood sample, commercially available, that has to be read after 18-24 hours; it detects the interleukins, in particular the gamma-interferon one, released by TH1-lymphocytes cooperating in cell-mediated response, if previously sensitized by the tuberculosis infection, when they are in cultures and are stimulated by mycobacterial antigens or haptens.

Differentiating the bovine active tuberculosis from the latent one is irrelevant, since the national and international compulsory eradication programmes do not allow the infected cattle to be raised, at any infection stage.

The skin test, requiring the official veterinarian's inspection on the farm twice in three days, is gradually replaced by the gamma interferon test, commercially available.

The kit arrangement, structured in order to have a rapid screening kit concerning the bovine tuberculosis, could make the compulsory controls, regulated by national and international sanitary programs, easier and cheaper; the kit could allow resource saving, both in tuberculosis-free areas and in tuberculosis infected areas, where the raised animals have to be regularly checked in order to stem the spread of the infection among the animal population and to reduce the risk of zoonotic transmission to humans.

Rapid diagnostic and user-friendly kits are also needed in the paratuberculosis or John's disease indirect diagnosis at early stage, when the infection caused by Mycobacterium avium subspecies paratuberculosis induces the cellular immune response in infected hosts, that excrete the pathogen contaminating the environment, transmitting the infection to the animal population; the humoral immune response detectable by standard serological tests develops in the following stages and it is less difficult to be detected. The paratuberculosis causes the productivity loss in livestock and it is a suspected zoonosis, probably involved in the Crohn's disease in humans.

In the state of the art, there are no systems for the arrangement of rapid diagnostic kits detecting the host cellular immune response to pathogenic microorganisms causing infections not detectable by antibody assays; moreover, equipment-free systems—based on the simultaneous detection of different cells exhibiting infection specific markers simultaneously quantifiable—are not reported; additionally, there are not solid phases for immumoenzymatic tests and ELISA useful for performing assays aimed to the simultaneous detection of both different markers and the different cells that exhibit them.

EP 0 154 687 describes a system having a first recess level that receives the liquid containing the cells, which covers each well; immunosorbent elements inserted into the wells detect the cell secreted proteins, yet surface cellular markers are not detectable. Moreover, the cell types could be identified carrying out further tests in the recovered liquid; this is incompatible with the arrangement of rapid assays.

WO 03/085401, from which the present invention derives, illustrates a system useful to arrange equipment-free and multiple immuno-enzymatic kits, since they include microplates or microstrips pre-filled with all the reagents to be used to carry out the assays. Its advantage is given by the use of microplates as mere reagent containers, while the device having immunosorbent protruding elements—that can be directly dipped in the sample collected in a tube and, then, in the microplate or microstrip wells containing the reagents—acts as solid phase. The device and the method described in WO 03/085401 and in the scientific publications that illustrate the experimental results obtained through experimenting with the system in applications concerning various fields are not apt to perform two types of multiple detection in the same analytical procedure, that are the identification of lymphocyte types that exhibit specific markers and the quantification of these different markers; these two types of multiiple detection have to be performed in order to meet the diagnostic requirements for tuberculosis, infection that mainly evocate the cellular immune response. The device and the method are suitable for the said diagnostic purposes when used in combination with the innovative extended well shaped solid phase, apt to hold immunosorbent elements in groups.

WO 2207/039400 illustrates a method and a kit based on the simultaneous detection of different cytokines produced in excess by lymphocytes sensitized by tuberculosis infection when they are stimulated by specific tuberculosis antigens, with which they are incubated for 6-72 hours; other tuberculosis antigens are used as control; the test procedure requires skilled personnel and equipped laboratories.

DE 103 33 545 illustrates a system composed of several modules forming several variously shaped recess levels, which are not apt to be set into a commercially available ELISA reader for the reading of the analytical results.

WO 96/02836 describes a computerized system incompatible with the purposes expressed herein, since it is suited for the nucleic acid tests in clinical sample where the pathogen is localized, resulting not applicable to the diagnosis of extra-pulmonary tuberculosis infection; the system has plate teeth arranged in a comb, which can be immersed into specific wells, whose size and format are not apt to be used in the present invention that includes steps where the entire device—having immuonosorbent elements protruding from a rod—is dipped in tubes in order to reduce the costs of the tuberculosis diagnostic kits and to spread diagnosis in developing Countries, where the infection rate is very high.

In U.S. 2007/237687 the described devices substantially differ from the extended wells arranged in microplates and microstrips because of the presence of bottlenecks that could interfere with the sample distribution and the liquid phase distribution—which have to be in contact with the 2 solid phases—in a uniform way, essential for providing a reliable diagnostic result.

U.S. 2010/083778 describes devices suited for retaining nonporous substrates which are simultaneously screened; they are absolutely unadapt for arranging the kits presented here, since they do not provide the separation into individual wells, which is essential for the correct diagnostic marker quantification, as illustrated here.

In DE 10 2008 0213 65, the equipment suited for receiving reagents via micro-cannulas differs from the invention ideated and presented here; in particular, it does not contain several extended wells in the same row, which are apt to arrange kits comprehending a control test—to be carried out in parallel to the sample analysis—in a single row of a microplate or in a microstrip entirely dedicated to a sample to be analyzed in order to detect different cell types, each of which exhibits different markers to be quantified.

The present invention aims to meet the need to realise a device, a method and a kit especially fitted for the different cellular or molecular marker detection joined with the simultaneous marker quantification on different cells or molecules that exhibit these markers; in particular, the so conceived kit is useful for the tuberculosis diagnosis, since it is easy to use and it requires minimal laboratory equipment.

### DESCRIPTION OF THE INVENTION

The present invention concerns:
devices in the shape of microplates or microstrips having wells with extended length apt to receive 3 or 4 or 6 immunosorbent elements protruding from a rod at the same modular distance of wells arranged in standard 8-wells microstrips or in 12-wells microstrips or in standard 96-wells microplates;
the method that uses these devices making two different shaped solid phases compete with each other; a solid phase is constituted by the extended wells, each of which immobilize a different cell or molecule in the examined sample; the other solid phase is constituted by immunosorbent elements protruding from a rod, each of which has been previously coated with one of the same markers to be detected in the sample; then, these immunosorbent elements are dipped into each extended well in groups of 3 or 4 or 6, after ligands for markers to be detected have been added in liquid phase in the extended wells; these ligands bind to the immunosorbent elements in a inversely proportional amount to the quantity of the markers of each type of cells or molecules immobilized on the proper extended well that competes for the binding; these ligands are simultaneously quantifiable by an immunoenzymatic assay carried out by dipping the rod with the immunosorbent elements into a tube containing the conjugate and, then, by dipping each single element into the wells of standard microplates or microstrips containing chromogenic substrate, where the spectrophotometric reading is finally performed;
kits suited for carrying out the above said method, using the above said devices with extended wells in order to obtain the simultaneously quantification of markers of different type of cells or molecules and the simultaneous detection of different types of cells or molecules exhibiting them, including immunoassay kits for a rapid tuberculosis diagnosis and paratuberculosis diagnosis at early stage, based on the quantification of different infection markers of different cooperating and cytolytic lymphocyte populations sensitized against different antigens or mycobacterial haptens.

The extended well shape of the devices is needed to perform the method which aims to obtain:

1. the different type of cells or molecules selective immobilization on each extended well surface—obtained by antibodies or through other systems—where different markers are to be simultaneously detected, so that the extended wells surface takes on the role of a solid phase for immuno-enzymatic tests and ELISA, becoming able to catch and to immobilize specific ligands in the liquid phase;

2. following capture by the immobilized cells and molecules, on the surface of extended wells, of specific ligands for each of the markers that are to be quantified simultaneously, which are inoculated in the extended wells as a standard amount in the liquid phase; these ligands captured by the cells immobilized on the surface of the extended wells cannot be measured directly by immuno-enzymatic tests and ELISA because of the interference of the cellular layer and the final spectrophotometric reading of the chromogenic reaction; the technical problem is solved by analytically detecting the proportional amount of the ligands that have been caught by a specifically sensitized second solid phase, that is dipped into the first one and starts to compete for the capture of the ligands in the liquid phase and where the ligands part one from another, so that they can be subsequently quantified one by one by immuno-enzymatic tests and ELISA in wells of standard microplates or microstrips; this second solid phase consists of immunosorbent elements protruding from a rod at the same modular distance of wells arranged in standard 8-wells or in 12-wells microstrips or in standard 96-wells microplates, where each element is sensitized by antibodies directed to a specific ligand and competes, for its capture, with one of the markers to be quantified in the sample;

3. the quantification of each single ligand specifically captured by the immunosorbent elements, obtained by immunoenzymatic tests and ELISA, which are performed by incubating the elements protruding from a rod in conjugate and subsequently in chromogenic substrate distributed in the single wells of standard microplates or microstrips, so that the analytical result is expressed in terms of optical density by spectrophotometric reading for each marker in each type of cells or molecules of the sample; the amount of each ligand captured by the specifically sensitized immunosorbent element is in an inverse proportion to the quantity of the respective marker of each type of cells or molecules of the sample, being detected by a competitive assay in which two differently shaped solid phases—the extended well and the elements protruding from a rod—are in competition for the capture of the same ligands in the liquid phase; this competitive assay differs from the ones reported in the state of the art, which make several molecules in the liquid phase compete for binding to the same solid phase and do not allow to predispose assays useful for the duplex determination of different markers and different cells or molecules which exhibit them; the quantification of each ligand captured by the respective immunosorbent element can be obtained by immuno-enzymatic tests or by other proper tests, such as chemiluminescence assays.

The inventive devices and method can be also used in the development of kits detecting the cellular immune response by an immuno-enzymatic assay that simultaneously quantifies the different lymphocyte markers in the tubercular and paratubercular infection, and simultaneously detects different cooperating and cytolytic lymphocyte populations that exhibit them, making achievable the rapid diagnosis of human tuberculosis, of bovine tuberculosis and of paratuberculosis in ruminants at early stage.

The present invention is thought to solve some problems in the diagnosis of the human tuberculosis, which is one of the most worldwide impact infection diseases:
to detect the cellular immune response characterized by the occurrence of cooperating T-lymphocytes and cytolytic T-lymphocytes that are specific for mycobacterium tuberculosis antigens, and to monitor its evolution during the disease stages and the therapy, due to the fact that the detection of the antibody response does not provide reliable diagnostic evidence;
to detect the cases of pulmonary infection, extra pulmonary infections and the latent infected people through a test of indirect diagnosis based on the detection of the cellular immune response;
to obtain the analytic result in a few hours, to be able to restrain infected patients in order to initiate a well-timed therapy;
the staff who carries out laboratory tests have to be trained in a short time period, compatibly with the developing countries' large-scale healthy programmes, where there is a higher rate of infections;
equipped and expensive laboratories, which are not widely spread in developing countries, must be not needed.

These diagnostic problems correspond to the need of solving technical problems in order to realize:
kits apt to detect and to quantify the occurrence of infection markers on different kinds of lymphocyte cells;
kits apt to detect the cellular immunological response indicating the mycobacterial infection regardless of its localization;
rapid kits, as the ones widely used for ELISA for other infections which can be diagnosed by antibody detection;
ready to use kits;
kits that can be used by minimal skilled personnel;
kits that can be used in minimal equipped laboratories.

In order to satisfy the above listed requirements—and adopting as diagnostic concept the simultaneously quantification of different lymphocyte markers detectable in mycobacterial infection and the identification of the different lymphocyte populations who exhibit them, such as cooperating CD4 T-lymphocytes and cytotoxic CD8 T-lymphocytes—the devices and the method ideated for their utilization, aimed to the simultaneously quantification of different cellular markers and to the simultaneous detection of the different cells that exhibit them, are used for the development of specific diagnostic kits that are outlined below in short.

Devices Apt to Kit Development

In the kit for a single sample analysis, the extended well device is constituted of microstrips with 3 extended wells, fit to hold 4 immunosorbent elements protruding from a rod at the same modular distance of wells arranged in 96-wells standard microplates; it is made of immunosorbent material useful to absorb monoclonal antibodies which specifically catch the sample CD4 T-lymphocytes or the CD8 T-lymphocytes, immobilizing them on 2 of the 3 extended wells, while the third one is not sensitized and serves as negative control.

In the 8 samples testing kit the extended well device is made of microplates containing 24 extended wells, 3 for each row from A to H; each extended well is fit to hold 4 immunosorbent elements protruding from a rod and the complete device is made of immunosorbent material. The elements protruding from a rod are made of immunosorbent material; they are 12 and they have previously been sensitized by the immobilization on each of them of one of the same markers that are to be quantified on every different type of cell in the sample.

Those elements are dipped into the extended wells which have captured the specific lymphocytes and where the liquid phase containing ligands for each marker was added. The two different shaped solid phases compete with the lymphocytes immobilized on the extended well—in the case that they have been sensitized by the tuberculosis infection—in capturing the ligands for the markers that are to be quantified.

Method

The method to detect and to quantify the different tuberculosis infection markers in different cooperating lymphocyte and cytolytic lymphocyte populations is based on the use of devices with extended wells in order to have the reaction of the competition between the two different shaped solid phases, through the following steps:
the different CD4 T-lymphocytes and CD8 T-lymphocytes of the sample bind themselves respectively to the surface of the extended wells due to the previously immobilization of specific catching agents;
in the following step, different biotinylated ligands to the infection lymphocytic markers to be quantified are added in the extended wells in the liquid phase, where the second solid phase is immediately immersed; this second solid phase is made of elements protruding from a rod, each of which is coated with a catcher, identical to one of the different markers that are to be detected on the lymphocytes;
therefore, the biotinylated ligands bind themselves to the proper element, in an inverse proportion to the amount of each marker exhibited on the CD4 T-lymphocyte or CD8 T-lymphocyte types immobilized on the first solid phase constituted of the extended well;
in a following analytic step, the device with the elements protruding from a rod is completely immersed into a vial containing the enzyme-streptavidin conjugate—which is cheaper to use than microplates and microstrips—due to the need of a single enzyme-streptavidin conjugate type, apt to detect the occurrence of the different biotinylated ligands caught by the elements; the conjugate amount caught by each element is in an inverse proportion to the quantity of the respective lymphocyte marker of the under testing sample;
finally, the elements are individually immersed into the wells of standard microplates or microstrips containing the chromogenic substrate, where the colorimetric reaction develops; this colorimetric reaction can be measured by the spectrophotometric reading.

Kits

The kits for the tuberculosis diagnosis based on the simultaneously quantification of different markers of infection present in the different lymphocyte populations are designed to essentially contain:
microstrips or microplates with extended wells, as previously described, on each of which a capture agent is immobilized for the specific lymphocyte type that has to be detected in the sample;
rods having protruding elements, each of which is sensitized by a capture agent that is the same of one of the different markers that are to be detected in each kind of lymphocyte type of sample immobilized on the extended wells;
the liquid phase containing a known standardized mix of ligands for the markers that are to be quantified on the different lynphocyte populations that are immobilized on the extended wells; these different lynphocyte populations come to a competition with the elements protruding from the rod in order to capture these ligands; these ligands are biotinylated;
a tube containing the horseradish peroxidase—streptavidin conjugate, where the rod with immunosorbent elements has to be immersed after their incubation in the liquid phase containing the ligands into the extended wells;
microstrips or microplates with chromogenic substrate distributed into the wells where the immunosorbent elements have to be individually immersed during the final analytical step.

The so designed kits allow to provide a solution suited for the main diagnostic problems that occur in the diagnosis of human and bovine tuberculosis, such as the extra-pulmonary localization of the infection, the time length required by the laboratory exams, the availability of well-equipped laboratories, expensive equipment and trained staff. The so designed kits allow an easier execution of the bovine tuberculosis prophylaxis operations in the veterinarian field. They also solve the problem of individuating the paratuberculosys infected ruminants at early stage, in order to reduce the spread of the infection in the livestock farming.

The ideated devices with extended wells and their use in the competitive reaction between two different shaped solid phases, that allows two multiple detection types such as the determination of types of cells which show specific markers and the different markers quantification, mutatis mutandis, are applicable to marker detection and quantification of cells, even antibody markers, making a selection among the different types of cells or molecules and the different antibody classes that exhibit them and that can be selected by an heterogenic sample through ready to use kits that do not need laboratory equipment and specialized staff to perform the test.

The size and the position of the extended wells, that are part of the ideated devices, can be selected according to the specific analytical and diagnostic needs, as described below.

The microplates or microstrips with extended wells have the size of standard 8 or 12-wells microstrips or of standard circular cross section 96-wells microplates; the adjustment consists of over-sizing wells along the length of the microstrips or along the directions of the rows or, alternatively, of the columns of microplates in order to obtain the desired gauge so that each microstrip, or each row or column of the microplate contains extended wells according to one of the methods described below:

2 extended wells—each of which is of the same lenght of 4 circular wells—can be contained in the microstrip of the size of a standard 8-wells microstrip and each extended well can contain 4 elements protruding from a rod, where these elements are set according to a modular distance that is the same of the modular arrangement of the wells of a standard 96-wells microplate or a standard 8 or 12-wells microstrip;

2 extended wells—each of which is of the length of 6 aligned wells of a standard 96-wells microplate—can be contained in the microstrip of the size of a standard 12-wells microstrip, and each extended well can contain 6 elements;

3 extended wells—each of which is of the same length of the 4 aligned wells of a standard 96-wells microplate—can be contained in the microstrip of the size of a standard 12-wells microstrip and each extended well can contain 4 elements;

4 extended wells—each of which is of the same length of the 3 aligned wells of a standard 96-wells microplate—can be contained in the microstrip of the size of a standard 12-wells microstrip and each extended well can contain 3 elements.

The microplate having the size of the standard 96-wells microplate can contain:
2 extended wells—each of which is of the same length of the 4 aligned wells of a standard 96-wells microplate—arranged in each of the 12 columns 1 up to 12 marked, for a total of 24 extended wells, each of which is apt to contain 4 elements;
2 extended wells—each of which is of the same length of the 6 aligned wells in a standard 96-wells microplate—arranged in each of the 8 rows A up to H marked, for a total of 16 extended wells, each of which is apt to contain 6 elements;
3 extended wells—each of which is of the same length of the 4 aligned wells in a standard 96-wells microplate—arranged in each of the 8 rows, A up to H marked, for a total of 24 extended wells, each of which is apt to contain 4 elements;
4 extended wells—each of which is of the same length of the 3 aligned wells in a standard 96-wells microplate—arranged in each of the 8 rows A up to H marked, for a total of 32 extended wells, each of which is apt to contain 3 elements.

The constituent material, the inner surface and the bottom shape of the extended wells can be chosen and arranged according to the specific analytical and diagnostic needs; for example, for the cell-cultures of cells whose specific surface markers are to be detected and quantified; or for the fixation of cells of thin sections obtained by microtome on frozen material or embedded material; or to absorb or to cultivate or to fix infected cells, in order to detect the occurrence of virus-specific or virus-induced antigens caused by wild types or vaccinal strains infections; or to catch different antibody classes whose specificity is to be detected; or to attract antibody or antigen coated paramagnetic beads after their bound with their respective molecular or cellular targets. These paramagnetic beads are hold on the bottom of the well by a magnet fixed outside. The list is given as an illustrative but not limitative example.

### BRIEF DESCRIPTION OF THE DRAWINGS

For a fuller understanding of the invention, reference should be made to the following detailed description, taken in connection with the accompanying drawings, in which:

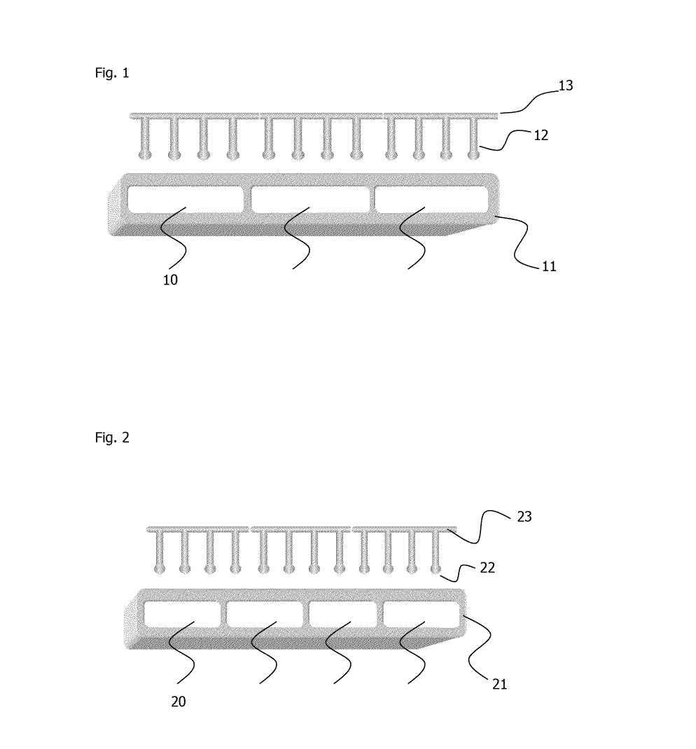

In FIG. 1, the microstrip (11) has the standard 12-wells microstrip dimension and it contains 3 extended wells of equal size; each extended well (10) has a length apt to receive 4 elements (12) of the device (13) having 12 elements protruding from a rod at a modular distance coinciding with the modular distance of the wells arranged in a standard 96-wells microplate and in a standard 12-wells microstrip.

In FIG. 2, the microstrip (21) has the standard 12-wells microstrip dimension and it contains 4 extended wells of equal size; each extended well (20) has a length apt to receive 3 elements (22) of the device (23) having 12 elements protruding from a rod at a modular distance coinciding with the modular distance of the wells arranged in a standard 96-wells microplate and in a standard 12-wells microstrip.

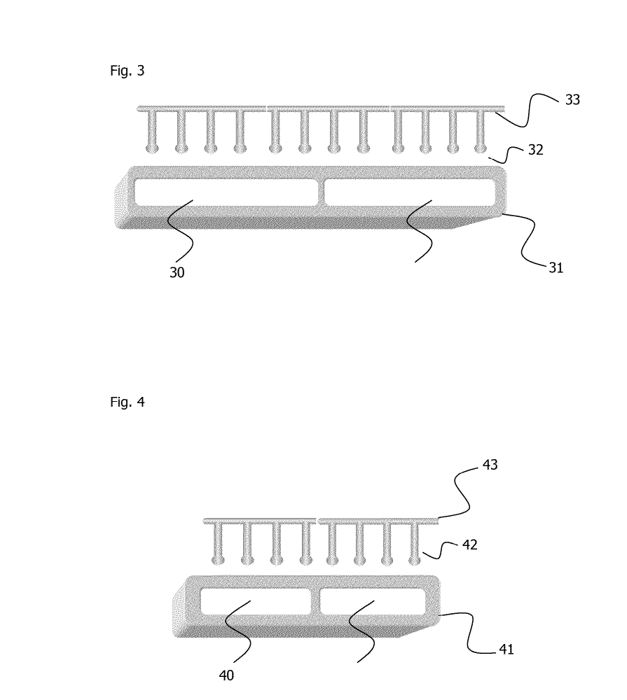

In FIG. 3, the microstrip (31) has the standard 12-wells microstrip dimension and it contains 2 extended wells of equal size; each extended well (30) has a length apt to receive 6 elements (32) of the device (33) having 12 elements protruding from a rod at a modular distance coinciding with the modular distance of the wells arranged in a standard 96-wells microplate and in a standard 12-wells microstrip.

In FIG. 4, the microstrip (41) has the standard 8-wells microstrip dimension and it contains 2 extended wells of equal size; each extended well (40) has a length apt to receive 4 elements (42) of the device (43) having 8 elements protruding from a rod at a modular distance coinciding with the modular distance of the wells arranged in a standard 96-wells microplate and in a standard 8-wells microstrip.

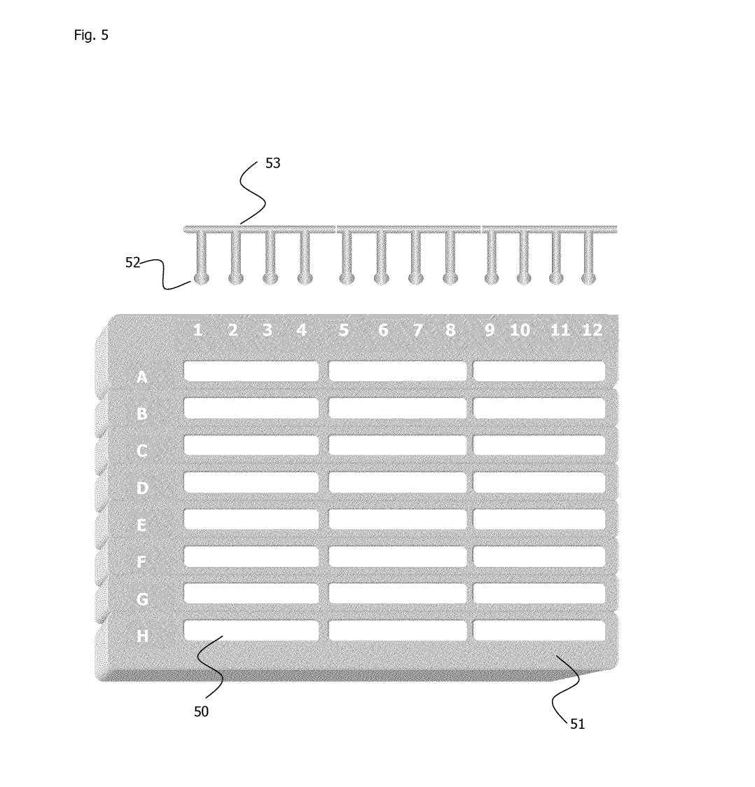

In FIG. 5, the microplate (51) has the standard 96-wells microplates dimension and it contains 24 extended wells of equal size; each extended well (50) has a length apt to receive 4 elements (52) of the device (53) having 12 elements protruding from a rod at a modular distance coinciding with the modular distance of the wells arranged in a standard 96-wells microplate.

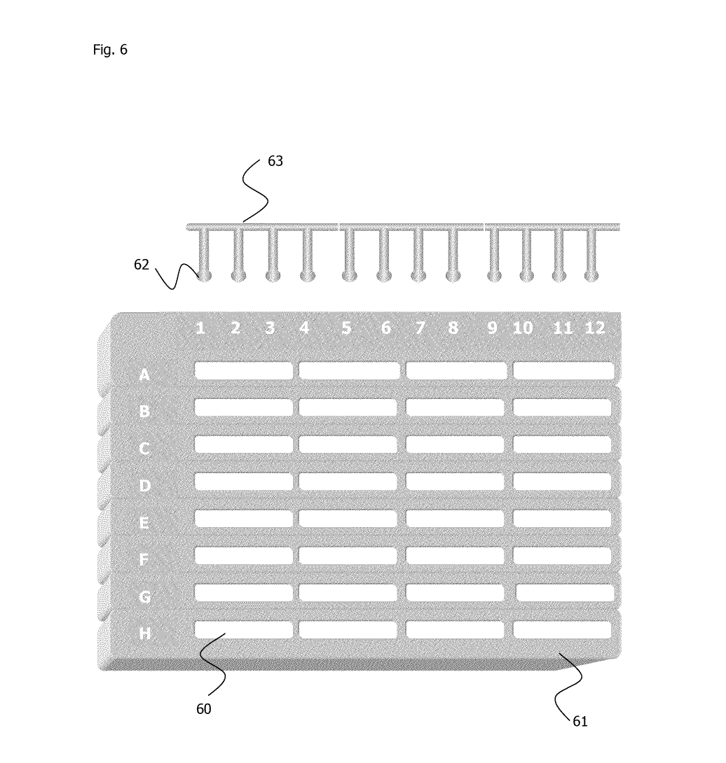

In FIG. 6, the microplate (61) has the standard 96-wells microplate dimension and it contains 32 extended wells of equal size; each extended well (60) has a length apt to receive 3 elements (62) of the device (63) having 12 elements protruding from a rod at a modular distance coinciding with the modular distance of the wells arranged in a standard 96-wells microplate.

### DETAILED DESCRIPTION OF THE PREFERRED EMBODIMENT

Kit Arrangement in the View of Rapid Tuberculosis Diagnosis Based on the Simultaneous Detection of Different Markers Exhibited on CD4 T-Lymphocytes and CD8 T-Lymphocytes

Arrangement examples of ready to use kits—based on the extended well devices and on the competitive reaction between two differently shaped solid phases—are described as follows; kits have been developed for the rapid diagnosis of human tuberculosis and for the rapid diagnosis of bovine tuberculosis joined with the paratuberculosis diagnosis at early stage. The kits permit development of an analytical report in a few hours and they are based on the cytolytic CD8 T-lymphocytes and the cooperating CD4 T-lymphocytes—comprising the TH1 lymphocytes cooperating in cellular response and TH2 lymphocytes cooperating in antibody response—detection, sensitized as a result of tuberculosis infection.

An exemplary arrangement of ready to use diagnostic kit for human tuberculosis, concerning a single sample assay, includes:
No. 1 3-extended-wells microstrip, each one with the extension of 4 circular wells of a standard 96-well microplate, sensitized as specified below:

well 1 is coated with anti-CD4 monoclonal antibody directed to the cooperating CD4 T-lymphocytes, which are bound in immune-complex on the well surface when whole blood sample is added;

well 2 is coated with anti-CD8 monoclonal antibody directed to the cytolytic CD8 T-lymphocytes, which are bound in immune-complex on the well surface when whole blood sample is added;

well 3 is not coated and acts as negative control to detect possible nonspecific reactions;
No. 1 disposable pipette for distributing 3 sample aliquots in the 3 extended wells;
No. 1 vial with dispenser, containing washing solution to wash the extended wells after the sample incubation;
No. 2 tubes containing washing solution, suitable to contain the entire device having immunosorbent elements protruding from a rod, which have to be immersed in the washing solution after the element incubation in the extended wells;
No. 3 vials containing the Mycobacterium tuberculosis biotinylated antigen standardized mix, including, but not limited to, ESAT-6, CFP-10 and TB 7.7;
No. 1 device having 12 immunosorbent elements protruding from a rod, coated with monoclonal antibodies (MAb) as specified below:

element 1: MAb for ESAT-6;

element 2: MAb for CFP-10;

element 3: MAb for TB 7.7;

element 4: no coated—negative control;

element 5: MAb to ESAT-6;

element 6: MAb for CFP-10;

element 7: MAb for TB 7.7;

element 8: no coated—negative control;

element 9: MAb to ESAT-6;

element 10: MAb for CFP-10;

element 11: MAb for TB 7.7;

element 12: no coated—negative control;
No. 1 tube containing conjugate, such as horseradish peroxidase-streptavidin;
No. 1 standard 12-wells microstrip;
   1 dropper vial containing the chromogenic substrate, such as trimethylbenzidine (TMB);
   blotting paper pads.

Alternatively, a standard no binding 96-wells microplate can be used, pre-filled with washing solution for the elements, conjugate and chromogenic substrate, distributed as follows:
washing solution in the wells of rows A, B, C, E, F and G;
conjugated streptavidin/enzyme into the wells of row D;
chromogenic substrate into the wells of row H.

1. LYMPHOCYTES CATCHING—Whole blood sample added with anticoagulant is distributed, in 1 mL aliquots, into each of the 3 extended wells arranged in the microstrip. During the incubation time, the catching of cooperating T-lymphocytes on the surface of extended well 1 and the catching of cytolytic T-lymphocytes on the surface of the extended well 2 occur.
2. COMPETITION BETWEEN 2 SOLID PHASES FOR THE BINDING TO THE BIOTINYLATED ANTIGENS—After incubation, the extended wells are washed 3 times with washing solution; in each well is then inoculated the biotinylated antigen ESAT-6, CFP-10 and TB 7.7 standardized mix, contained in the proper vial.

The device having 12 coated elements is positioned onto the microstrip, so that 4 elements are immersed in each extended well; incubation occurs.

In extended well 1 the antibodies coating the device elements bind the biotinylated antigens and compete with the CD4 T-lymphocytes sensitized by tuberculosis infection previously caught on the extended well surface, while in extended well 2 they compete with the CD8 T-lymphocytes sensitized by tuberculosis infection previously caught on the extended well surface.

After incubation, the amount of biotinylated antigens bound in immuno-complex on each specific element is inversely proportional to the presence of markers exhibited by the sensitized lymphocytes previously caught on the extended well surface, that bind part of the same antigens through specific surface receptors, evocated by the tuberculosis infection.

3. ELEMENTS BINDING CONJUGATE—At the end of incubation, the device having the immunosorbent elements protruding from a rod is lifted up and immersed in one of the two tubes containing washing solution. After elements are dried on blotting paper pads, the entire device is immersed in the tube containing the conjugate; then incubation occurs.

Alternatively, if the reagents are pre-distributed in microplate, the elements are introduced into the wells of row A in a standard 96-well microplate, so that each element is immersed into a well containing washing solution; then the elements are dried on blotting paper. Washing step is performed also in the wells of rows B and C, followed by the drying of the elements. The elements are then immersed in the wells of row D, containing the conjugate; incubation occurs.

4. CHROMOGENIC REACTION—After the incubation step occurred in the conjugate, the device having the immunosorbent elements protruding from a rod is lifted up and immersed in the remaining tube containing washing solution. After the drying of the elements on blotting paper, they are singularly dipped into the microstrip wells where the chromogenic substrate was previously distributed.

Alternatively, if the reagents are pre-distributed in microplate, the elements are introduced into the wells of rows E, F and G; then they are dried and finally dipped into the wells of row H, containing the chromogenic substrate.

After the incubation, the elements are lifted up and the solution Optical Density (OD)—in the wells where the chromogenic reaction was developed—is read by spectrophotometer or ELISA reader. In case of equipment lacking, test results can be read visually, comparing each color of the test wells with the color of negative control wells.

5. INTERPRETATION OF RESULTS—The detected O.D. is inversely proportional to the amount of lymphocytes, respectively CD4 cooperating or CD8 cytotoxic, which are present in the blood sample, specific for the proper tuberculosis antigen; the interpretation of test results has to be made in relation to the O.D. measured in correspondence of elements 9, 10 and 11, which, since the absence of competitor lymphocytes in extended well 3, bind the maximum amount of each biotinylated antigen, while elements 4, 8 and 12 act as specificity control.

The positive control is set up to calculate the calibration curve useful for the quantitative interpreting of the analytical results, including the cutoff point. In relation to the scientific progress, different markers and different cell types may be replaced or added among those that the kit can detect and quantify, about which the above description is only given as an example.

The similar kit arrangement for the diagnosis of bovine tuberculosis joined with the diagnosis of paratuberculosis at early stage can be obtained including the haptens:
bovine tuberculin acting as tuberculosis infection marker to be quantified on different CD4 and CD8 T-lymphocytes;
avian tuberculin, acting as cross-reacting antigen in the aim to highlight false positive results evocated by the temporary infection with Mycobacterium avium;
Johnina, acting as paratuberculosis infection marker to be quantified on different CD4 and CD8 T-lymphocytes.

Ready to use kits for many samples have to be arranged in order to comprise at least:
microstrips having 3 extended wells, each of which has a length apt to receive 4 coated immunosorbent elements, or microplates having 3 extended wells arranged in 8 rows marked marked with letters A, B, C, D, E, F, G and H in a standard microplate, coated for each type of diagnosis, as above specified;
vials containing biotinylated antigen or hapten standardized mix;
devices having 12 coated elements, as above described for the diagnosis;
tubes containing washing solution, tubes containing the conjugate, standard 96-well microplates or a sufficient number of standard 12-well microstrips where the chromogenic substrate is distributed;
blotting paper pads for the drying of the elements.

In case of availability of pre-filled microplates, in place of tubes containing conjugate and washing solution, standard 96-wells microplates or a sufficient number of standard 12-wells microstrips pre-filled with conjugate and as many pre-filled with chromogenic substrate have to be arranged in the kit;
dispenser vials containing washing solution;
frame to hold and to simultaneously move the 12 immunosorbent element devices from the microplate containing the conjugate to the microplate containing the chromogenic substrate.

In this case, the manual test execution is carried out, after incubating blood samples in the extended wells and the washing step, in each of the extended wells the selected mycobacteria antigen or hapten standardized mix is inoculated; then the frame containing the 8 devices having 12 coated immunosorbent elements is positioned, moving simultaneously the devices, so that the immunosorbent elements are:
dipped in the extended wells and incubated;
washed and dried;
immersed in the standard wells containing the conjugate and incubated;
washed and dried;
immersed in the standard wells containing the chromogenic substrate and incubated;
lifted up for the reading of the results performed by ELISA reader in the latter wells, in which the chromogenic reaction develops.

The easy operative procedures might allow a potential robotization of the kits arranged as described above.

In the preceding specification, all documents, acts, or information disclosed does not constitute an admission that the document, act, or information of any combination thereof was publicly available, known to the public, part of the general knowledge in the art, or was known to be relevant to solve any problem at the time of priority.

The disclosures of all publications cited above are expressly incorporated herein by reference, each in its entirety, to the same extent as if each were incorporated by reference individually.

While there has been described and illustrated specific embodiments of the present invention, it will be apparent to those skilled in the art that variations and modifications are possible without deviating from the broad spirit and principle of the invention. It is also to be understood that the following claims are intended to cover all of the generic and specific features of the invention herein described, and all statements of the scope of the invention which, as a matter of language, might be said to fall therebetween.

What is claimed is:

1. A immuno-enzymatic tests device, comprising
at least one microplate or microstrip further comprising a plurality of elongated test wells, wherein the elongated test well is dimensioned to receive 3, 4, or 6 of the immunosorbent protruding elements
an immobilization ligand disposed as a solid phase in the interior of each test well, where each well contains a immobilization ligand adapted to immobilize a different type of cell contained in the sample;
at least one rod, further comprising
a test member; and
a plurality of immunosorbent protruding elements disposed at a modular distance from the test member, where the immunosorbent protruding elements are disposed at a distance that coincides with the position of a well on the at least one microplate or microstrip;
where each of the plurality of immunosorbent protruding elements is coated with a capture molecule corresponding to a detection antigen; and
where the antigen is adapted to detect different types of cells or molecules of the sample that have been immobilized in said wells, and antigens correlating to the capture molecule coat the immunosorbent elements protruding from the rod.

2. The device of claim 1, wherein the at least one microplate or microstrip is a standard microplate or microstrip.

3. The device of claim 1, wherein the capture molecules are a plurality of monoclonal antibodies.

4. The device of claim 3, wherein the monoclonal antibodies are MAb against ESAT-6, MAb against CFP-10, or MAb against TB 7.7.

5. The device of claim 1, wherein at least one of the immunosorbent protruding elements is not coated to serve as a negative control.

6. The device of claim 1, wherein the immobilization ligand is anti-CD4 monoclonal antibody, or anti-CD8 monoclonal antibody.

7. The device of claim 6, wherein at least one of the wells is not coated to serve as a negative control.

8. A method for detecting different types of cells and molecules, comprising the steps of:
providing a test blood sample;
providing an immuno-enzymatic tests device, where the device further comprises:
at least one microplate or microstrip further comprising a plurality of elongated test wells, wherein the elongated test well is dimensioned to receive 3, 4, or 6 of the immunosorbent protruding elements;
an immobilization ligand disposed as a solid phase in the interior of each test well, where each well contains a immobilization ligand adapted to immobilize a different type of cell contained in the sample;
at least one rod, further comprising
a test member;
a plurality of immunosorbent protruding elements disposed at a modular distance from the test member, where the immunosorbent protruding elements are disposed at a distance that coincides with the position of a well on the at least one microplate or microstrip;
where each of the plurality of immunosorbent protruding elements is coated with a capture molecule corresponding to a detection antigen;
where the antigen is adapted to detect different types of cells or molecules of the sample that have been immobilized in said wells, and antigens correlating to the capture molecule coat the immunosorbent elements protruding from the rod;
immobilizing a type of cell or molecule of the test blood sample in the elongated test well by incubating the test blood sample in liquid phase in the elongated test well;
washing the elongated test well;
adding a biotinylated antigen to each of the extended wells in the liquid phase, wherein the biotinylated antigen is adapted to detect the infection lymphocytic marker to be quantified;
immersing the immunosorbent protruding elements into the elongated test well;
incubating the immunosorbent protruding elements, wherein the antigen binds the immunosorbent elements in an inversely proportional amount to the quantity of the catcher of the different types of cells or molecules immobilized on the respective extended well;
placing the immunosorbent protruding elements into a container containing conjugate;
placing the immunosorbent protruding elements into chromogenic substrate; and
performing a spectrophotometric reading on the immunosorbent protruding elements.

9. The method of claim 8, test wells are washed 3 times with washing solution.

10. The method of claim 8, wherein the immunosorbent protruding elements are completely immersed into a vial containing the conjugate.

11. The method of claim 10, wherein the conjugate is enzyme-streptavidin conjugate.

12. The method of claim 8, wherein the immunosorbent protruding elements are individually immersed into wells of standard microplates or microstrips containing the chromogenic substrate.

13. The method of claim 8, wherein the capture molecules are MAb against ESAT-6, MAb against CFP-10, or MAb against TB 7.7.

14. The method of claim 13, wherein at least one of the immunosorbent protruding elements is not coated to serve as a negative control.

15. The method of claim 8, wherein the immobilization ligand is anti-CD4 monoclonal antibody, or anti-CD8 monoclonal antibody.

16. The method of claim 15, wherein at least one of the wells is not coated to serve as a negative control.

17. The method of claim 8, wherein the chromogenic substrate is trimethylbenzidine.

18. A kit for detecting different types of cells and molecules, comprising
at least one microplate or microstrip further comprising a three elongated test wells, wherein the elongated test well is dimensioned to receive 3, 4, or 6 of the immunosorbent protruding elements;
wherein a first elongated test well is coated with anti-CD4 antibodies for immobilizing the cooperating T-lymphocytes;
wherein a second elongated test well is coated with anti-CD8 antibodies for immobilizing the cytolytic T-lymphocytes;
wherein a third elongated test wells is not coated and acts as negative control;
vials containing a detection antigen, wherein the detection antigen is mycobacterial antigen or hapten;
at least one rod, further comprising
a test member;
a plurality of immunosorbent protruding elements disposed at a modular distance from the test member, where the immunosorbent protruding elements are disposed at a distance that coincides with the position of a well on the at least one microplate or microstrip;
where each of the plurality of immunosorbent protruding elements is coated with a capture molecule corresponding to a detection antigen;
where the antigen is adapted to detect different types of cells or molecules of the sample that have been immobilized in said wells, and antigens correlating to the capture molecule coat the immunosorbent elements protruding from the rod;
a wash solution; and
a chromogenic substrate.

19. The kit of claim 17, further comprising no-binding standard 96-well microplates arranged in 8 rows each of which has 12 wells, or containers as tubes or trays.

20. The kit of claim 17, wherein the chromogenic substrate is trimethylbenzidine.
